(19) United States
(12) Patent Application Publication
(10) Pub. No.: US 2002/0182172 A1
(43) Pub. Date: Dec. 5, 2002

(54) Water-soluble polymer conjugates of triazine derivatives

(75) Inventors: Michael David Bentley, Huntsville, AL (US); Xuan Zhao, Huntsville, AL (US); Robert Shorr, Edison, NJ (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Shearwater Corporation

(21) Appl. No.: 09/999,197

(22) Filed: Nov. 30, 2001

Related U.S. Application Data
(60) Provisional application No. 60/250,483, filed on Nov. 30, 2000.

(51) Int. Cl. A61K 31/785; C08F 216/06

(52) U.S. Cl. 424/78.36; 528/423; 525/54.2; 525/56; 525/259

(57) ABSTRACT

The present invention provides water-soluble polymer conjugates of triazine derivatives using water soluble and non-peptidic polymer backbones, such as poly(ethylene glycol). The invention includes conjugates made using mPEG, bifunctional PEG, branched or multi-arm PEG, and forked PEG. The invention further includes a method of forming such conjugates and a method of treating conditions responsive to triazine derivatives using the conjugates.

### CROSS-REFERENCE TO RELATED APPLICATIONS

This application claims the benefit of Provisional Application Serial No. 60/250,483, filed Nov. 30, 2000, which is incorporated herein by reference in its entirety.

### FIELD OF THE INVENTION

This invention relates to water-soluble polymer conjugates of biologically active molecules. More specifically, the present invention is directed to polymer conjugates of triazine-based active agents and to methods for making and administering such conjugates.

### BACKGROUND OF THE INVENTION

Triazine derivatives have considerable potential as drugs, and several triazine-based compounds have been shown to be effective as anti-tumor agents. For example, a triazine derivative, trimelamol, has shown promising activity as an anticancer drug.

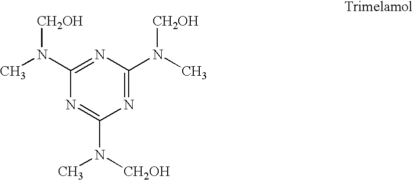

In clinical trials against ovarian cancer (I. R. Judson, et al., Cancer Research, 49:5475-5479, 1989; I. R. Judson, et al., Br. J. Cancer, 63:311-313, 1991), safety and a degree of efficacy were demonstrated for trimelamol, but formulation problems associated with low aqueous solubility and a high propensity for trimelamol dimerization and precipitation resulted in discontinuation of the trials. Synthetic analogues of trimelamol have been prepared, but they were difficult to purify and the analogues exhibited only a marginal improvement in stability (U.S. Pat. No. 5,854,244).

Thus, although many triazine derivatives demonstrating antitimor activity have been synthesized (Matsuno, T., et al., Chem Pharm Bull, 2000, 48(11): 1778-81; Abdel-Rahman R M, et al., Pharmazie, 1999, 54(9):667-71), such compounds tend to be chemically unstable (i.e., prone to degradation, dimerization, hydrolysis), making both chemical modification and/or formulation particularly difficult. Moreover, triazine anticancer drugs, while shown to be effective in both in vitro and in vivo evaluations, tend to be highly toxic. Thus, an approach is needed for maintaining or enhancing the antitumor efficacy of certain triazine agents, while reducing the adverse side effects and increasing the chemical stability of such agents.

### SUMMARY OF THE INVENTION

The present invention is based upon the discovery of new water-soluble polymer conjugates of triazine-based compounds, and a unique synthetic approach for preparing such conjugates which avoids the problems of triazine derivative dimerization and instability. Specifically, the invention provides, in one aspect, water-soluble polymer conjugates of certain triazine derivatives, such as N-alkyl-N-(hydroxymethyl) aminotriazines. The conjugates have greatly improved water solubility and stability in solution compared to trimelamol.

The polymer conjugates of the invention comprise at least one water soluble and non-peptidic polymer backbone covalently attached at a non-heteroatom position of (i) an s-triazine ring (i.e., 1,3,5-triazine) of a triazine derivative, or (ii) an as-triazine ring (i.e., 1,2,4-triazine) of a triazine derivative. Preferably, the non-peptidic polymer conjugate comprises a polymer backbone covalently attached at only one non-heteroatom position within the triazine ring of the derivative. In one embodiment, the polymer conjugates of the invention comprise a water soluble and non-peptidic polymer backbone, such as poly(ethylene glycol), bonded to the following structure:

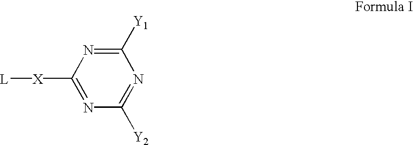

wherein:

L is the point of attachment to the polymer backbone;

X is a linker, such as O or NR₂, wherein R₂ is H, C1-6alkyl, or substituted C1-6alkyl (e.g., CH₂OH); and

Y₁ and Y₂ are each independently amino, substituted amino, C1-6alkyl, substituted C1-6alkyl, aryl, or substituted aryl.

In one embodiment, Y₁ and Y₂ are each NRR₁, wherein R is C1-6alkyl (e.g., methyl), substituted C1-6alkyl, or an electron withdrawing group (e.g., —CH₂CF₃ or —CH₂C≡CH), and R₁ is H, C1-6alkyl, or substituted C1-6alkyl (e.g., CH₂OH).

Suitable polymer backbones include poly(alkylene glycol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), and copolymers, terpolymers, and mixtures thereof.

The polymer conjugates of the invention may be formed using linear polymer backbone starting materials, such as mPEG or bifunctional PEG, or multi-arm polymer backbones. More specifically, the invention includes heterobifunctional polymer conjugates wherein one terminus of the polymer backbone is attached to the triazine derivative moiety and the other terminus is functionalized with a different moiety. Additionally, the invention includes homobifunctional polymer conjugates, wherein both termini of the polymer backbone are bonded to triazine derivatives.

Also forming part of the present invention is a method of making a polymer conjugate of a triazine derivative which differs significantly from the customary approach of conjugate formation in which a polymer is reacted directly with a reactive moiety of an active drug. Certain triazine derivatives are not particularly amenable to the direct conjugation approach, due to their instability in solution. In an effort to overcome this problem, the inventors have devised a synthetic methodology in which the polymer is first attached to a relatively stable precursor of the triazine drug molecule to form a pegylated triazine intermediate, which is then further modified in one or more synthetic steps to form the active triazine derivative portion of the conjugate. Thus, the active drug portion of the conjugate is synthesized after chemical attachment of the water soluble polymer portion rather than before. The presence of the polymer portion of the triazine intermediate is believed to have a stabilizing effect during subsequent synthesis of the active triazine derivative portion of the conjugate.

Specifically, the invention includes, in another aspect, a method of forming the polymer conjugates of the invention in which the polymer backbone is first conjugated to a precursor triazine structure, such as cyanuric halide, followed by modification of the triazine skeleton to form the active triazine moiety. This approach allows purification of the product of each synthetic step to be accomplished in high yield by, for example, selective precipitation of the product from an appropriate organic solvent or solvent mixture, such as diethyl ether, isopropanol, or mixtures thereof. Moreover, this route avoids the problems of triazine dimerization and is highly selective for mono-polymer substitution within the triazine ring.

The invention also provides for the use of these conjugates for the treatment of diseases responsive to triazine derivatives, including various types of cancer. The method of treatment comprises administering to a mammal a therapeutically effective amount of a polymer conjugate of the present invention.

### DETAILED DESCRIPTION OF THE INVENTION

The present invention now will be described more filly hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art.

I. Definitions

The terms “functional group”, “active moiety”, “activating group”, “reactive site”, “chemically reactive group” and “chemically reactive moiety” are used in the art and herein to refer to distinct, definable portions or units of a molecule. The terms are somewhat synonymous in the chemical arts and are used herein to indicate the portions of molecules that perform some function or activity and are reactive with other molecules. The term “active,” when used in conjunction with functional groups, is intended to include those functional groups that react readily with electrophilic or nucleophilic groups on other molecules, in contrast to those groups that require strong catalysts or highly impractical reaction conditions in order to react (i.e., “non-reactive” or “inert” groups). For example, as would be understood in the art, the term “active ester” would include those esters that react readily with nucleophilic groups such as amines. Exemplary active esters include N-hydroxysuccinimidyl esters or 1-benzotriazolyl esters. Typically, an active ester will react with an amine in aqueous medium in a matter of minutes, whereas certain esters, such as methyl or ethyl esters, require a strong catalyst in order to react with a nucleophilic group.

The term “linkage” or “linker”(e.g., the X moiety described below) is used herein to refer to an atom, groups of atoms, or bonds that are normally formed as the result of a chemical reaction. A linker of the invention typically links the connecting moieties, such as a polymer backbone and a triazine derivative, via one or more covalent bonds. Hydrolytically stable linkages means that the linkages are substantially stable in water and do not react to any significant degree with water at useful pHs, e.g., under physiological conditions for an extended period of time, perhaps even indefinitely. Hydrolytically unstable or degradable linkages means that the linkages are degradable in water or in aqueous solutions, including for example, blood. Enzymatically unstable or degradable linkages means that the linkage can be degraded by one or more enzymes. As understood in the art, PEG and related polymers may include degradable linkages in the polymer backbone or in the linker connecting the polymer backbone and a triazine derivative.

The terms “alkyl” refers to hydrocarbon chains typically ranging from about 1 to about 12 carbon atoms in length, and includes straight and branched chains. The hydrocarbon chains may be saturated or unsaturated. The term “substituted alkyl” refers to an alkyl group substituted with one or more non-interfering substituents, such as, but not limited to, C3-C6 cycloalkyl, e.g., cyclopropyl, cyclobutyl, and the like; acetylene; cyano; alkoxy, e.g., methoxy, ethoxy, and the like; lower alkanoyloxy, e.g., acetoxy; hydroxy; carboxyl; amino; lower alkylamino, e.g., methylamino; ketone; halo, e.g. chloro or bromo; phenyl; substituted phenyl, and the like.

“Aryl” means one or more aromatic rings, each of 5 or 6 core carbon atoms. Multiple aryl rings may be fused, as in naphthyl or unfused, as in biphenyl. Aryl rings may also be fused or unfused with one or more cyclic hydrocarbon, heteroaryl, or heterocyclic rings.

“Substituted aryl” is aryl having one or more non-interfering groups as substituents. For substitutions on a phenyl ring, the substituents may be in any orientation (i.e., ortho, meta or para).

“Non-interfering substituents” are those groups that yield stable compounds. Suitable non-interfering substituents or radicals include, but are not limited to, halo, C1-C10 alkyl, C2-C10 alkenyl, C2-C10 alkynyl, C1-C10 alkoxy, C7-C12 aralkyl, C7-C12 alkaryl, C3-C10 cycloalkyl, C3-C10 cycloalkenyl, phenyl, substituted phenyl, toluoyl, xylenyl, biphenyl, C2-C12 alkoxyalkyl, C7-C12 alkoxyaryl, C7-C12 aryloxyalkyl, C6-C12 oxyaryl, C1-C6 alkylsulfinyl, C1-C10 alkylsulfonyl, —(CH₂)ₘ—O—(C1-C10 alkyl) wherein m is from 1 to 8, aryl, substituted aryl, substituted alkoxy, fluoroalkyl, heterocyclic radical, substituted heterocyclic radical, nitroalkyl, —NO₂, —CN, —NRC(O)—(C1-C10 alkyl),—C(O)—(C1-C10 alkyl), C2-C10 thioalkyl,—C(O)O—(C1-C10 alkyl), —OH, —SO₂, ═S, —COOH, —NR, carbonyl, —C(O)—(C1-C10 alkyl)-CF₃, —C(O)—CF₃, —C(O)NR₂, —(C1-C10 alkyl)—S—(C6-C 12 aryl), —C(O)—(C6-C12 aryl), —(CH₂)ₘ—O—(CH₂)ₘ—O—(C 1-10 alkyl) wherein each m is from 1 to 8, —C(O)NR, —C(S)NR, —SO₂NR, —NRC(O)NR, —NRC(S)NR, salts thereof, and the like. Each R as used herein is H, alkyl or substituted alkyl, aryl or substituted aryl, aralkyl, or alkaryl.

“Substituted amino” refers to amino groups of the formula NR₃R₄ wherein at least one of R₃ and R₄ is a non-interfering substituent as defined above, such as C1-6alkyl or substituted C1-6alkyl. “Polyolefinic alcohol” refers to a polymer comprising an olefin polymer backbone, such as polyethylene, having multiple pendant hydroxyl groups attached to the polymer backbone. An exemplary polyolefinic alcohol is polyvinyl alcohol.

As used herein, “non-peptidic” refers to a polymer backbone substantially free of peptide linkages. However, the polymer backbone may include a minor number of peptide linkages spaced along the length of the backbone, such as, for example, no more than about 1 peptide linkage per about 50 monomer units.

“Cyanuric halide” refers to an s-triazine or as-triazine ring having at least one halogen atom covalently attached to a non-heteroatom position of the triazine ring. Preferably, the cyanuric halide molecule has three halogen atoms attached to non-heteroatom positions of the triazine ring, such as cyanuric chloride.

A “polymer conjugate of a triazine derivative” refers to a water soluble and non-peptidic polymer backbone covalently attached to a triazine derivative as defined herein, wherein the triazine ring portion of the conjugate is absent (i) halo substituents, and (ii) a covalently attached protein. That is to say, the polymer-substituted triazine derivatives of the present invention are not protein modifiers, but rather themselves are drug conjugates.

II. The Polymer Conjugate

The present invention is based upon the discovery of certain novel polymer conjugates of triazine derivatives. The conjugates of the invention overcome the chemical instability and insolubility problems of the parent triazine derivatives, and are preferably prepared by a synthetic approach which avoids the problems of low yields and dimerization of the parent compound and results in high yields of the conjugate precursor. The conjugates themselves and their method of synthesis will now be more fully described.

The polymer conjugates of the invention comprise at least one water soluble and non-peptidic polymer backbone covalently attached through a linkage to a non-heteroatom position of (i) an s-triazine ring of a triazine derivative, or (ii) an as-triazine ring of a triazine derivative. Preferably, the non-peptidic polymer conjugate comprises a polymer backbone covalently attached at only one non-heteroatom (i.e., non-nitrogen) position within the triazine ring of the derivative.

The term “triazine derivative” is intended to encompass any structure comprising a 1,3,5-triazine or 1,2,4-triazine ring. As used herein, the term includes triazine structures comprising fused rings, such as benzotriazine rings. The triazine derivatives may be substituted at any of the heteroatom positions and/or substituted at one or more of the remaining non-heteroatom positions of the triazine ring structure that are not covalently bonded to the polymer backbone. Exemplary substituents for the non-heteroatom positions of the triazine ring include amino, substituted amino (e.g. alkylamino and dialkylamino), aryl (e.g., phenyl), substituted aryl (e.g., phenyl substituted with, for example, one or more halogen atoms).

In addition to trimelamol described above, other examples of specific triazine compounds that can form the triazine derivative portion of the polymer conjugate of the present invention include altretamine (2,4,6-dimethylamino-1,3,5-triazine), lamotrigine (3,4-diamino-6-(2,3 -dichlorophenyl)-1,2,4-triazine), and tirapazamine (3-amino-1,2,4-benzotriazine-1,4-dioxide), all of which are shown below. Altretamine is an antitumor drug with demonstrated activity against refractory ovarian cancer (Damia et al., Clin. Pharmacokinet., 1995, 28(6): 439-449). Due to its poor water solubility, it is typically administered orally. Tirapazamine is a lead compound in a class of bioreductive benzotriazine compounds that exhibits the ability to selectively kill hypoxic tumor cells (Koch, Cancer Research, 1993, 53: 3992-3997). Lamotrigine is an anticonvulsant useful in treating epilepsy and has also shown promise in the treatment and control of pain associated with diabetic neuropathy and SUNCT (Short-lasting, Unilateral, Neuralgiform headache attacks with Conjunctival Injection and Tearing) Syndrome (Eisenberg, et al., Neurology, 2001, 57(3):505-509; D'Andrea, et al., Neurology, 2001, 57(9): 1723-1725).

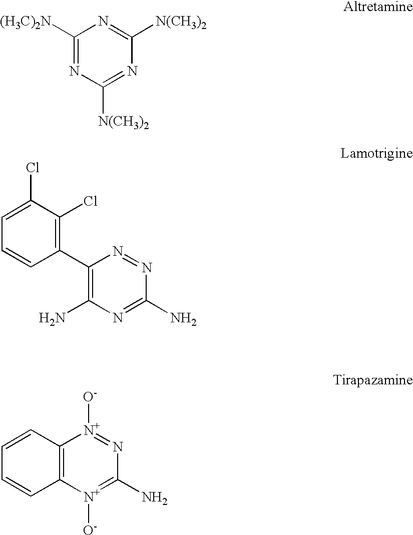

With respect to lamotrigine, the polymer backbone may be attached to the triazine ring at either of the amino-substituted positions. Alternatively, the polymer backbone may be attached to any available carbon atom on the phenyl ring. With regard to tirapazamine, the polymer backbone may be attached to any available carbon atom on the fused ring structure or at the amino-substituted position.

In one particular embodiment, the invention is directed to polymer conjugates of triazine derivatives comprising a water soluble and non-peptidic polymer backbone bonded to the following structure referred to herein as Formula I:

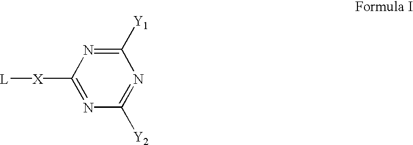

wherein:

L is the point of attachment to the polymer backbone;

X is a linker, such as O or NR_{2,} wherein R₂ is H, C1-6alkyl, or substituted C1-6alkyl (e.g., CH₂OH); and

Y₁ and Y₂ are each independently amino, substituted amino, C1-6alkyl, substituted C1-6alkyl, aryl, or substituted aryl.

In yet another specific embodiment, Y₁ and Y₂ are each NRR₁, wherein R is C1-6alkyl (e.g., methyl), substituted C1-6alkyl, or an electron withdrawing group (e.g., —CH₂CF₃ or —CH₂C≡CH), and R₁ is H, C1-6alkyl, or substituted C1-6alkyl. When the triazine derivative attached to the polymer backbone is altretamine, both R and R₁ are C1-6alkyl, specifically methyl. When the triazine derivative attached to the polymer backbone is trimelamol, R is methyl and R₁ is —CH₂OH.

A. The Polymer Backbone

The water-soluble and non-peptidic polymer backbone of Formula I can be poly(ethylene glycol) (i.e. PEG). However, it should be understood that other related polymers are also suitable for use in the practice of this invention and that the use of the term PEG or poly(ethylene glycol) is intended to be inclusive and not exclusive in this respect. The term PEG includes poly(ethylene glycol) in any of its linear, branched or multi-arm forms, including alkoxy PEG, bifunctional PEG, forked PEG, branched PEG, pendant PEG, or PEG with degradable linkages therein, to be more fully described below.

PEG, in any of the forms described herein, is typically clear, colorless, odorless, soluble in water, stable to heat, inert to many chemical agents, does not hydrolyze or deteriorate (unless specifically designed to do so), and is generally non-toxic. Poly(ethylene glycol) is considered to be biocompatible, which is to say that PEG is capable of coexistence with living tissues or organisms without causing harm. More specifically, PEG is substantially non-immunogenic, which is to say that PEG does not tend to produce an immune response in the body. When attached to a molecule having some desirable function in the body, such as a biologically active triazine derivative of the present invention, the PEG tends to mask the agent and can reduce or eliminate any immune response so that an organism can tolerate the presence of the agent. PEG conjugates tend not to produce a substantial immune response or cause clotting or other undesirable effects. PEG having the formula—CH₂CH₂O—(CH₂CH₂O)ₙ—CH₂CH₂—, where n is from about 3 to about 4000, typically from about 3 to about 2000, is one useful polymer in the practice of the invention. PEGs having a number average molecular weight of from about 100 Da to about 100,000 Da, preferably about 350 Da to 40,000 Da are particularly useful as the polymer backbone.

In one form useful in the present invention, free or non-bound PEG is a linear polymer terminated at each end with hydroxyl groups:

HO—CH₂CH₂O—(CH₂CH₂O)ₙ—CH₂CH₂—OH

The above polymer, alpha-,omega-dihydroxylpoly(ethylene glycol), can be represented in brief form as HO—PEG—OH where it is understood that the —PEG—symbol represents the following structural unit:

—CH₂CH₂O—(CH₂CH₂O)ₙ—CH₂CH₂—

where n typically ranges from about 3 to about 4000. A linear polymer backbone of this type is used as a starting material in Example 7.

Another type of PEG useful in forming the conjugates of the invention is methoxy-PEG—OH, or MPEG in brief, in which one terminus is the relatively inert methoxy group, while the other terminus is a hydroxyl group that is subject to ready chemical modification. The structure of mPEG is given below.

CH₃O—(CH₂CH₂O)ₙ—CH₂CH₂—OH

where n is as described above. The use of polymer backbones in the form of mPEG is exemplified in Examples 1-5 and 8.

Random or block copolymers of ethylene oxide and propylene oxide, shown below, are closely related to PEG in their chemistry, and can also be used as the polymer backbone of the conjugates of the invention.

HO—CH₂CHRO(CH₂CHRO)ₙCH₂CHR—OH

wherein each R is independently H or CH₃, and n is as described above.

The polymer backbones may also comprise a branched structure, typically having a central branching core moiety and a plurality of polymer chains, preferably linear polymer chains, linked to the central core. In one embodiment, PEG is used in a branched form prepared, for example, by addition of ethylene oxide to various polyol central core structures, such as glycerol, glycerol oligomers, pentaerythritol and sorbitol. Any polyol providing a plurality of hydroxyl groups available for conjugation to polymer chains may be used in the present invention. The polyol branching core structure provides about 3 to about 100 available hydroxy groups (typically about 3 to about 20) such that the branched polymer structure will comprise about 3 to about 100 polymer chains. The branched poly(ethylene glycol) molecules of this type can be represented in general form as R(-PEG-OH)ₘ in which R is derived from a central core moiety, such as glycerol, glycerol oligomers, or pentaerythritol, and m represents the number of arms, typically about 3 to about 20. Use of a branched PEG structure formed using pentaerythritol as a central core is exemplified in Example 6. The central core moiety can also be derived from any of a number of amino acids, such as lysine, wherein the central core moiety typically provides two or more sites, e.g., amino groups, for attachment of polymer chains. Multi-armed PEG molecules, such as those described in U.S. Pat. No. 5,932,462, which is incorporated by reference herein in its entirety, can also be used as the polymer backbone. The polymer backbones described in U.S. Pat. No. 5,932,462 are discussed in greater detail below in connection with Formula Ie.

The polymer backbone may alternatively comprise a forked PEG. An example of a forked PEG is represented by PEG—YCHZ₂, where Y is a linking group and Z is an activated terminal group linked to CH by a chain of atoms of defined length. International Application No. PCT/US99/05333, the contents of which are incorporated by reference herein, discloses various forked PEG structures for use in one embodiment of the invention. The chain of atoms linking the Z functional groups to the branching carbon atom serve as a tethering group and may comprise, for example, alkyl chains, ether chains, ester chains, amide chains and combinations thereof. The Z functional groups can be used in the present invention to react with the triazine derivative and form a linkage between the triazine derivative and the polymer backbone. A forked polymer embodiment is discussed in greater detail below in connection with Formula Id.

The polymer backbone may comprise a pendant PEG molecule having reactive groups, such as carboxyl, covalently attached along the length of the PEG backbone rather than at the end of the PEG chain. The pendant reactive groups can be attached to the PEG backbone directly or through a linking moiety, such as alkylene.

In addition to the above-described forms of PEG, the polymer can also be prepared with one or more weak or degradable linkages in the backbone, including any of the above described polymers. For example, PEG can be prepared with ester linkages in the polymer backbone that are subject to hydrolysis. As shown below, this hydrolysis results in cleavage of the polymer into fragments of lower molecular weight:

—PEG—CO₂—PEG—+H₂O)→—PEG—CO₂H+HO—PEG—

Similarly, a polymer backbone can be covalently attached to a biologically active agent, such as a triazine derivative, through a weak or degradable linkage moiety. For example, ester linkages formed by the reaction of PEG carboxylic acids or activated PEG carboxylic acids with alcohol groups on a biologically active agent generally hydrolyze under physiological conditions to release the agent.

Other hydrolytically degradable linkages, useful as either a degradable linkage within a polymer backbone or as a degradable linkage connecting a polymer backbone to a biologically active agent, include carbonate linkages; imine linkages resulting, for example, from reaction of an amine and an aldehyde (see, e.g., Ouchi et al., Polymer Preprints, 38(1):582-3 (1997), which is incorporated herein by reference.); phosphate ester linkages formed, for example, by reacting an alcohol with a phosphate group; hydrazone linkages which are typically formed by reaction of a hydrazide and an aldehyde; acetal linkages that are typically formed by reaction between an aldehyde and an alcohol; orthoester linkages that are, for example, formed by reaction between a formate and an alcohol; peptide linkages formed by an amine group, e.g., at an end of a polymer such as PEG, and a carboxyl group of a peptide; and oligonucleotide linkages formed by, for example, a phosphoramidite group, e.g., at the end of a polymer, and a 5′ hydroxyl group of an oligonucleotide.

It is understood by those skilled in the art that the term poly(ethylene glycol) or PEG represents or includes all the above forms of PEG.

Many other polymers are also suitable for the invention. Polymer backbones that are non-peptidic and water-soluble, with from 2 to about 300 termini, are particularly useful in the invention. Examples of suitable polymers include, but are not limited to, other poly(alkylene glycols), such as poly(propylene glycol) (“PPG”), copolymers of ethylene glycol and propylene glycol and the like, poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), such as described in U.S. Pat. No. 5,629,384, which is incorporated by reference herein in its entirety, and copolymers, terpolymers, and mixtures thereof. Although the number average molecular weight of each chain of the polymer backbone can vary, it is typically in the range of from about 100 Da to about 100,000 Da, often from about 350 Da to about 40,000 Da. These polymers may be linear, or may be in any of the above described forms (e.g., branched, forked, and the like).

Those of ordinary skill in the art will recognize that the foregoing list for substantially water soluble and non-peptidic polymer backbones is by no means exhaustive and is merely illustrative, and that all polymeric materials having the qualities described above are contemplated.

B. Linkage Between Polymer Backbone and Triazine Derivative

The linkage between the triazine derivative and the polymer backbone, such as the X moiety in Formula I above, results, at least in part, from the reaction of a functional group attached to the polymer backbone with the triazine derivative molecule. The specific linkage will depend on the type of functional group utilized. Assuming the polymer backbone is relatively simple in structure without a forking end group or a branched structure such as described in U.S. Pat. No. 5,932,462, and possesses at least one hydroxyl terminus for attachment to the triazine derivative, X will be O. Similarly, if a relatively simple polymer backbone is functionalized with an amine group, X will be NR₂, wherein R₂ is H, C1-6alkyl, or CH₂OH. When certain multi-arm, branched or forked polymer backbones are used, the X moiety will be relatively more complex and may include a longer linkage structure. For example, as shown below in one exemplary “forked” polymer embodiment (Formula Id), the X moiety comprises the —X₁—(W)ₚ—CH—Y′-linkage between the terminus of the polymer backbone and the triazine derivative moiety. The overall X linkage is intended to encompass any linkage between the polymer backbone and the triazine derivative molecule having an overall length of from 1 to about 20 atoms, preferably 1 to about 10 atoms.

C. Exemplary Conjugate Structures

More specific structural embodiments of the conjugates of the invention will now be described, all of which are intended to be encompassed by the structure of Formula I above. The specific structures shown below are presented as exemplary structures only, and are not intended to limit the scope of the invention. Other triazine derivative structures, such as those referred to above, could be substituted for the specific 1,3,5-triazine derivatives shown in Formulas Ia-Id. For instance, 1,2,4-triazine derivatives could be used as the triazine derivative portion of the conjugates.

In one embodiment, a substantially linear version of the polymer conjugate of the invention has the following structure:

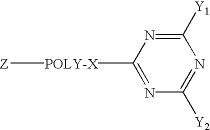

POLY is a water soluble and non-peptidic polymer backbone;

Z is a capping group as described below;

X is a linker, such as O or NR₂, wherein R₂ is H, C1-6alkyl, or substituted C1-6alkyl (e.g., CH₂OH); and

Y₁ and Y₂ are each independently amino, substituted amino, C1-6alkyl, substituted C1-6alkyl, aryl, or substituted aryl.

The Z moiety can be any suitable capping group for polymers of the type described herein. For example, the Z capping group can be a relatively inert group, such as an alkoxy group (e.g. methoxy or ethoxy). Alternatively, the Z moiety can be a reactive functional group, optionally in protected form, such as hydroxyl, protected hydroxyl, active ester (e.g. N-hydroxysuccinimidyl ester or 1-benzotriazolyl ester), active carbonate (e.g. N-hydroxysuccinimidyl carbonate and 1-benzotriazolyl carbonate), acetal, aldehyde, aldehyde hydrates, alkenyl, acrylate, methacrylate, acrylamide, active sulfone, amine, protected amine, hydrazide, protected hydrazide, thiol, protected thiol, carboxylic acid, protected carboxylic acid, isocyanate, isothiocyanate, maleimide, vinylsulfone, dithiopyridine, vinylpyridine, iodoacetamide, epoxide, glyoxals, diones, mesylates, tosylates, or tresylate.

As would be understood in the art, the term “protected” refers to the presence of a protecting group or moiety that prevents reaction of the chemically reactive functional group under certain reaction conditions. The protecting group will vary depending on the type of chemically reactive group being protected and the reaction conditions employed. For example, if the chemically reactive group is an amine or a hydrazide, the protecting group can be selected from the group of tert-butyloxycarbonyl (t-Boc) and 9-fluorenylmethoxycarbonyl (Fmoc). If the chemically reactive group is a thiol, the protecting group can be orthopyridyldisulfide. If the chemically reactive group is a carboxylic acid, such as butanoic or propionic acid, or a hydroxyl group, the protecting group can be benzyl or an alkyl group such as methyl, ethyl, or tert-butyl. Other protecting groups known in the art may also be used in the invention, see for example, Greene, T. W., et al., PROTECTIVE GROUPS IN ORGANIC SYNTHESIS, 2nd ed., John Wiley & Sons, New York, N.Y. (1991).

Specific examples of terminal functional groups for the polymer backbones of the invention include N-succinimidyl carbonate (see e.g., U.S. Pat. Nos. 5,281,698, 5,468,478), amine (see, e.g., Buckmann et al. Makromol.Chem. 182:1379 (1981), Zaplipsky et al. Eur. Polym. J. 19:1177 (1983)), hydrazide (See, e.g., Andresz et al. Makromol. Chem. 179:301 (1978)), succinimidyl propionate and succinimidyl butanoate (see, e.g., Olson et al. in Poly(ethylene glycol) Chemistry & Biological Applications, pp 170-181, Harris & Zaplipsky Eds., ACS, Washington, D.C., 1997; see also U.S. Pat. No. 5,672,662), succinimidyl succinate (See, e.g., Abuchowski et al. Cancer Biochem. Biophys. 7:175 (1984) and Joppich et al. Macrolol. Chem. 180:1381 (1979), succinimidyl ester (see, e.g., U.S. Pat. No. 4,670,417), benzotriazole carbonate (see, e.g., U.S. Pat. No. 5,650,234), glycidyl ether (see, e.g., Pitha et al. Eur. J. Biochem. 94:11 (1979), Elling et al., Biotech. Appl. Biochem. 13:354 (1991), oxycarbonylimidazole (see, e.g., Beauchamp, et al., Anal. Biochem. 131:25 (1983), Tondelli et al. J. Controlled Release 1:251 (1985)), p-nitrophenyl carbonate (see, e.g., Veronese, et al., Appl. Biochem. Biotech., 11:141 (1985); and Sartore et al., Appl. Biochem. Biotech., 27:45 (1991)), aldehyde (see, e.g., Harris et al. J. Polym. Sci. Chem. Ed. 22:341 (1984), U.S. Pat. No. 5,824,784, U.S. Pat. No. 5,252,714), maleimide (see, e.g., Goodson et al. Bio/Technology 8:343 (1990), Romani et al. in Chemistry of Peptides and Proteins 2:29 (1984)), and Kogan, Synthetic Comm. 22:2417 (1992)), orthopyridyl-disulfide (see, e.g., Woghiren, et al. Bioconj. Chem. 4:314 (1993)), acrylol (see, e.g., Sawhney et al., Macromolecules, 26:581 (1993)), vinylsulfone (see, e.g., U.S. Pat. No. 5,900,461). All of the above references are incorporated herein by reference.

Homobifunctional polymer conjugates corresponding to Formula Ia above, wherein a central polymer backbone connects two triazine derivatives, are also included in the present invention, wherein Z has the structure:

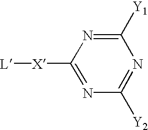

wherein X′ is a linker, L′ is the point of attachment to POLY, and Y₁ and Y₂ are as defined above. In a preferred embodiment, both X and X′ are O and POLY is poly(ethylene glycol).

The invention also includes multi-arm polymer conjugates having, for example, 3 to about 100 termini. An example of a multi-arm or branched conjugate having a plurality of polymer arms attached to a central core molecule has the structure:

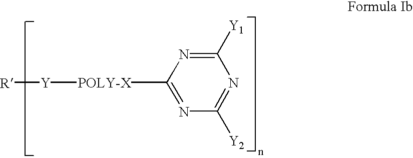

wherein:

n is an integer from 3 to about 100, preferably about 3 to about 20;

R′ is a central core molecule;

X and Y are each independently selected linkers, such as O or NR₂, wherein R₂ is H, C1-6alkyl or CH₂OH;

each POLY is an independently selected water-soluble and non-peptidic polymer backbone; and

Y₁ and Y₂ are as defined above.

The central core molecule, R, is preferably derived from a molecule selected from the group consisting of polyols, such as glycerol, glycerol oligomers, pentaerythritol or sorbitol, polyamines, such as polylysine or other polyamino acids, and molecules having a combination of alcohol and amine groups. Alternatively, the R moiety may comprise a dendrimer of the type described in U.S. Pat. No. 5,830,986, which is incorporated by reference in its entirety, such as polyamidoamine dendrimers, poly(propylenimine) dendrimers and the like. Preferably, the molecular weight of R is less than about 2,000 Da. The central core molecule is derived from a molecule having n number of functional sites capable of attaching to n number of polymer backbones, POLY, via a linkage, Y. The ability to attach a plurality of polymer backbones to the central core molecule increases the loading capacity of the polymer, which is particularly useful for biologically active agents having relatively low activity.

One specific example of a multi-arm conjugate of the invention has the structure:

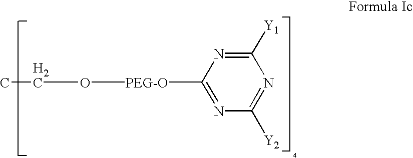

wherein PEG is poly(ethylene glycol) having an average molecular weight from about 100 Da to about 100,000 Da, and Y₁ and Y₂ are as defined above.

A specific example of a “forked” polymer conjugate of the invention is shown below:

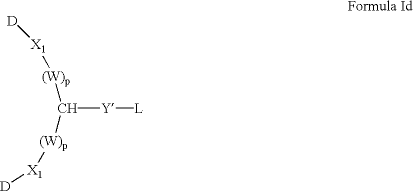

wherein X₁ and Y′ are independently selected linkers, such as such as 0 or NR₂, wherein R₂ is H, C1-6alkyl or CH₂OH; L is the point of bonding to the polymer backbone, each p is independently 0 or 1, and each W is a tethering group, such as —(CH₂)ₘ—, —(CH₂)ₘ—O—, —O—(CH₂)ₘ—, —(CH₂)ₘ—O₂C—CH₂CH₂—,and —(CH₂)ₘ—O—(CH₂)ᵣ—, wherein m and r are independently 1-10, and each D is a triazine derivative, such as a triazine derivative having the structure:

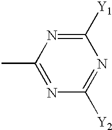

wherein Y₁ and Y₂ are as defined above.

In another embodiment, the polymer conjugate is formed using a branched polymer backbone of the type described in U.S. Pat. No. 5,932,462, wherein the polymer backbone has the structure:

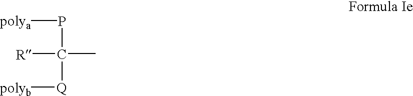

wherein:

polyₐ and poly_{b} are water-soluble and non-peptidic polymer backbones, such as methoxy poly(ethylene glycol);

R″ is a nonreactive moiety, such as H, methyl or a water-soluble and non-peptidic polymer backbone; and

P and Q are nonreactive linkages. In a preferred embodiment, the branched polymer backbone comprises methoxy poly(ethylene glycol) disubstituted lysine.

III. Preparation of Polymer Conjugates

Another aspect of the invention is an indirect method for forming the above-described polymer conjugates. In the method, the polymer backbone is not, as is the customary approach, conjugated directly to an active drug moiety. Instead, the polymer is conjugated to a commercially-available precursor to the active drug to form a pegylated triazine drug precursor, which is then further modified by reactions with the triazine skeleton to build the active drug portion of the molecule.

This approach was developed after discovering that direct conjugation of a water-soluble polymer to the active triazine derivative, trimelamol, was incredibly difficult, resulting in low yields and a complicated mixture of products due in part to hydrolysis of the trimelamol. The exemplary triazine drug precursor employed, cyanuric chloride, has previously been used as a linker to link polyethylene glycol to active proteins such as interferon (Abuchowski, et al., J. Biol. Chem., 252, 3578 (1977); U.S. Pat. No. 5,342,940), but has heretofore, not been used as a synthetic precursor to generate an active drug moiety, such as a triazine-based antitumor compound.

In general, the method involves reacting a water soluble polymer as described above with a cyanuric halide or an equivalent thereof, to form a reactive triazine intermediate having a polymer arm substituted at one of the non-heteroatom positions of the triazine ring. Typically the polymer, e.g., PEG, is activated at one terminus for displacement of/substitution for one of the halogen atoms of the cyanuric halide. Preferred are reactive moieties such as the corresponding alkoxide salt of hydroxy-terminated PEG, or amino-terminated PEG, although any of a number of reactive groups could be employed, as could be readily determined by one of skill in the art. Due to the nature of the triazine ring, polymer substitution typically occurs only at one position, thus making this a highly selective synthesis route. That is to say, di-and tri-polymer substituted triazines are not typically formed to any significant extent, making this a high yield synthetic approach. Moreover, polymer attachment at only a single site within the triazine ring structure is generally preferred, due to the small molecule nature of the active agents forming the basis of present invention. Representative reactions coupling illustrative PEG backbones to a cyanuric chloride are shown in Examples 1, 4, 6, 7, and 8.

Subsequent to attachment of the polymer backbone, the cyanuric halide intermediate is then modified, in either one or a series of chemical modification steps, to introduce functional groups at the halide positions within the triazine ring corresponding to the active triazine derivative portion of the conjugate. In preferred embodiments of the invention, such functional groups are selected from the group consisting of amino, substituted amino (e.g. alkylamino and dialkylamino), aryl (e.g., phenyl), substituted aryl (e.g., phenyl substituted with, for example, one or more halogen atoms).

More specifically, the method comprises providing a water soluble and non-peptidic polymer backbone bonded to a functional group reactive with a cyanuric halide. The functional group is preferably hydroxyl or amino. For example, when forming polymer-derivatized 1,3,5-triazine derivatives, the polymer backbone is preferably reacted with a cyanuric halide having the structure:

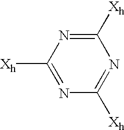

wherein each Xₕ is halogen, preferably chlorine.

Typically, the reaction between the polymer backbone and the cyanuric halide occurs in the presence of a suitable solvent, such as toluene, tetrahydrofuran, or dioxane, preferably in anhydrous form. In one embodiment, the terminal functional group of the water-soluble and non-peptidic polymer backbone is hydroxyl, which is converted to the corresponding alkoxide by reaction with a strong base, such as n-butyl or t-butyl lithium, followed by reaction with the cyanuric halide.

The thus-formed dihalotriazine intermediate having a polymer arm attached thereto is then further modified to replace the remaining halogen or other substituents on the triazine ring with those corresponding to an active triazine derivative. For instance, the dihalotriazine polymer intermediate is reacted with an amine, e.g., an alkyl amine such as methyl amine, to form a diamino-substituted triazine polymer conjugate bonded to the structure:

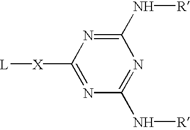

wherein R′ is alkyl, preferably C1-6alkyl, and L and X are as defined above. Exemplary reactions replacing the remaining halides with substituted amino groups are set forth in Examples 2, 4, and 6-8.

In this instance, the two halo functionalities in the polymer-derivatized dihalotriazine intermediate molecule are replaced with identical functional groups, which may undergo additional chemical modifications, depending upon the structure of the desired triazine derivative product. In instances where the functional groups substituted on the triazine ring in the final triazine derivative are dissimilar, stepwise introduction of the desired functional groups is employed.

Typically, conversion of the polymer-derivatized dihalo substituted triazine precursor to the corresponding conjugate of an active triazine derivative, such as by reaction with an alkyl amine, occurs in the presence of a suitable solvent, such as toluene, tetrahydrofuran, dioxane, acetonitrile, methylene chloride, or chloroform.

The triazine derivative polymer conjugate may also be further modified, for example, at the introduced amino groups, by reaction with aqueous formaldehyde in the presence of an alkali metal carbonate, such as potassium carbonate, to form a polymer conjugate having disubstituted amino groups on the triazine ring, as illustrated below:

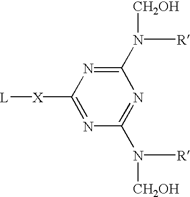

wherein L, X and R′ are as defined above. Exemplary reactions with formaldehyde are set forth in Examples 3, 5, and 6-8.

The above-described method of synthesis is particularly advantageous because an intermediate polymer conjugate is formed in the first step, which greatly enhances/simplifies purification of the final product, since the polymer product of each synthetic step can be collected as a precipitate from suitable solvents, such as diethyl ether, isopropanol, or mixtures thereof. Thus, due to the ease of separation of the polymer-attached triazine compounds formed in each reaction step, purification can readily occur at each stage of the overall synthesis rather than only at the end of the process.

IV. Compositions/Formulations Comprising Polymer Conjugate

The chemically-modified triazine derivatives provided herein will typically possess one or more of the following characteristics. The triazine derivative conjugates of the invention, in addition to having a high purity/homogeneity, maintain at least a measurable degree of specific activity. That is to say, a triazine conjugate in accordance with the invention will possesses anywhere from about 2% to about 100% or more of the specific activity of the unmodified, parent triazine compound. Such activity may be determined using a suitable in-vivo or in-vitro antitumor model, depending upon the known activity of the particular triazine parent compound. For instance, the antitumor activity of the conjugate may be readily determined using standard Leukemia, Lung, Breast, and CNS anticancer evaluations, e.g., employing human cancer cell lines or murine leukemia cell lines, and measuring the activity of the conjugate against the activity of the unmodified parent triazine compound. In general, a triazine conjugate of the invention will possess a specific activity of at least about 2%, 5%, 10%, 15%, 25%, 30%, 40% or more relative to that of the unmodified parent triazine, when measured in a suitable antitumor model, such as those well known in the art.

The polymer conjugates of the invention may be administered per se or in the form of a pharmaceutically acceptable salt. If used, a salt of the polymer conjugate should be both pharmacologically and pharmaceutically acceptable, but non-pharmaceutically acceptable salts may conveniently be used to prepare the free active compound or pharmaceutically acceptable salts thereof and are not excluded from the scope of this invention. Such pharmacologically and pharmaceutically acceptable salts can be prepared by reaction of the polymer conjugate with an organic or inorganic acid, using standard methods detailed in the literature. Examples of useful salts include, but are not limited to, those prepared from the following acids: hydrochloric, hydrobromic, sulfuric, nitric, phosphoric, maleic, acetic, salicyclic, p-toluenesulfonic, tartaric, citric, methanesulphonic, formic, malonic, succinic, naphthalene-2-sulphonic and benzenesulphonic, and the like. Also, pharmaceutically acceptable salts can be prepared as alkaline metal or alkaline earth salts, such as sodium, potassium, or calcium salts of a carboxylic acid group.

The present invention also provides pharmaceutical formulations or compositions, both for veterinary and for human medical use, which comprise one or more polymer conjugates of the invention or a pharmaceutically acceptable salt thereof, with one or more pharmaceutically acceptable carriers, and optionally any other therapeutic ingredients, stabilizers, or the like. The carrier(s) must be pharmaceutically acceptable in the sense of being compatible with the other ingredients of the formulation and not unduly deleterious to the recipient thereof. The compositions of the invention may also include polymeric excipients/additives or carriers, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, surfactants (e.g., polysorbates such as “TWEEN 20” and “TWEEN 80”, and pluronics such as F68 and F88, available from BASF), sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the invention are listed in “Remington: The Science & Practice of Pharmacy”, 19ₜₕ ed., Williams & Williams, (1995), and in the “Physician's Desk Reference”, 52^{nd} ed., Medical Economics, Montvale, N.J. (1998), and in “Handbook of Pharmaceutical Excipients”, Third Ed., Ed. A. H. Kibbe, Pharmaceutical Press, 2000.

The conjugates of the invention may be formulated in compositions including those suitable for oral, rectal, topical, nasal, ophthalmic, or parenteral (including intraperitoneal, intravenous, subcutaneous, or intramuscular injection) administration. The compositions may conveniently be presented in unit dosage form and may be prepared by any of the methods well known in the art of pharmacy. All methods include the step of bringing the active agent or compound (i.e., the polymer conjugate) into association with a carrier that constitutes one or more accessory ingredients. In general, the compositions are prepared by bringing the active compound into association with a liquid carrier to form a solution or a suspension, or alternatively, bring the active compound into association with formulation components suitable for forming a solid, optionally a particulate product, and then, if warranted, shaping the product into a desired delivery form. Solid formulations of the invention, when particulate, will typically comprise particles with sizes ranging from about 1 nanometer to about 500 microns. In general, for solid formulations intended for intravenous administration, particles will typically range from about 1 nm to about 10 microns in diameter.

The amount of triazine derivative conjugate in the formulation will vary depending upon the specific triazine derivative employed, its activity in conjugated form, its molecular weight, and other factors such as dosage form, target patient population, and other considerations, and will generally be readily determined by one skilled in the art. The amount of conjugate in the formulation will be that amount necessary to deliver a therapeutically effective amount of triazine derivative to a patient in need thereof to achieve at least one of the therapeutic effects associated with the triazine derivative, e.g., oncolytic activity. In practice, this will vary widely depending upon the particular conjugate, its activity, the severity of the condition to be treated, the patient population, the stability of the formulation, and the like. Compositions will generally contain anywhere from about 1% by weight to about 99% by weight triazine derivative conjugate, typically from about 2% to about 95% by weight conjugate, and more typically from about 5% to 85% by weight conjugate, and will also depend upon the relative amounts of excipients/additives contained in the composition. More specifically, the composition will typically contain at least about one of the following percentages of triazine conjugate: 2%, 5%, 10%, 20%, 30%, 40%, 50%, 60%, or more by weight.

Compositions of the present invention suitable for oral administration may be presented as discrete units such as capsules, cachets, tablets, lozenges, and the like, each containing a predetermined amount of the active agent as a powder or granules; or a suspension in an aqueous liquor or non-aqueous liquid such as a syrup, an elixir, an emulsion, a draught, and the like.

A tablet may be made by compression or molding, optionally with one or more accessory ingredients. Compressed tablets may be prepared by compressing in a suitable machine, with the active compound being in a free-flowing form such as a powder or granules which is optionally mixed with a binder, disintegrant, lubricant, inert diluent, surface active agent or dispersing agent. Molded tablets comprised with a suitable carrier may be made by molding in a suitable machine.

A syrup may be made by adding the active compound to a concentrated aqueous solution of a sugar, for example sucrose, to which may also be added any accessory ingredient(s). Such accessory ingredients may include flavorings, suitable preservatives, an agent to retard crystallization of the sugar, and an agent to increase the solubility of any other ingredient, such as polyhydric alcohol, for example, glycerol or sorbitol.

Formulations suitable for parenteral administration conveniently comprise a sterile aqueous preparation of the active compound, which can be formulated to be isotonic with the blood of the recipient.

Nasal spray formulations comprise purified aqueous solutions of the active agent with preservative agents and isotonic agents. Such formulations are preferably adjusted to a pH and isotonic state compatible with the nasal mucous membranes.

Formulations for rectal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye.

Topical formulations comprise the active compound dissolved or suspended in one or more media such as mineral oil, petroleum, polyhydroxy alcohols or other bases used for topical formulations. The addition of other accessory ingredients as noted above may be desirable.

Further, the present invention provides liposomal formulations of the polymer conjugates or salts thereof. The technology for forming liposomal suspensions is well known in the art. Aqueous soluble polymer conjugates of the invention, or salts thereof, can be incorporated into lipid vesicles using conventional liposome technology. In such an instance, due to the water solubility of the conjugate or salt, the conjugate or salt will be substantially entrained within the hydrophilic center or core of the liposomes. The lipid layer employed may be of any conventional composition and may either contain cholesterol or may be cholesterol-free. The liposomes that are produced may be reduced in size, for example, through the use of standard sonication and homogenization techniques. The liposomal formulations containing the polymer conjugates of the invention may be lyophilized to produce a lyophilizate which may be reconstituted with a pharmaceutically acceptable carrier, such as water, to regenerate a liposomal suspension.

Pharmaceutical formulations are also provided which are suitable for administration as an aerosol, by inhalation. These formulations comprise a solution or suspension of the desired polymer conjugate or a salt thereof. The desired formulation may be placed in a small chamber and nebulized. Nebulization may be accomplished by compressed air or by ultrasonic energy to form a plurality of liquid droplets or solid particles comprising the conjugates or salts thereof.

V. Method of Using the Polymer Conjugates

The polymer conjugates of the invention can be used to treat any condition responsive to triazine derivatives in mammals, including humans. A preferred condition for treatment is cancer. The method of treatment comprises administering to the mammal a therapeutically effective amount of a composition or formulation containing polymer conjugate of a triazine derivative as described above. The therapeutically effective dosage amount of any specific conjugate will vary somewhat from conjugate to conjugate, patient to patient, and will depend upon factors such as the condition of the patient, the loading capacity of the polymer conjugate, and the route of delivery. As a general proposition, a dosage from about 0.5 to about 20 mg/kg body weight, preferably from about 1.0 to about 5.0 mg/kg, will have therapeutic efficacy. When administered conjointly with other pharmaceutically active agents, even less of the polymer conjugate may be therapeutically effective.

The polymer conjugate may be administered once or several times a day. The duration of the treatment may be once per day for a period of from two to three weeks and may continue for a period of months or even years. The daily dose can be administered either by a single dose in the form of an individual dosage unit or several smaller dosage units or by multiple administration of subdivided dosages at certain intervals. Possible routes of delivery include buccally, subcutaneously, transdermally, intramuscularly, intravenously, orally, or by inhalation.

VI. Experimental

The following examples in which mPEG and other forms of PEG are used are given to illustrate the invention, but should not be considered in limitation of the invention. Additional forms of PEG and similar polymers that are useful in the practice of the invention are encompassed by the invention as discussed above. Further, while the examples below utilize specific triazine precursors and triazine derivative structures, the invention is not limited to the specific triazine structures exemplified.

All PEG reagents referred to in the appended examples are available from Shearwater Corporation of Huntsville, Al. All NMR data was generated by a 300 MHz NMR spectrometer manufactured by Bruker.

### EXAMPLE 1

### Synthesis of 2-mPEGyloxy₅ₖ-4,6-dichloro-1,3,5-triazine (I)

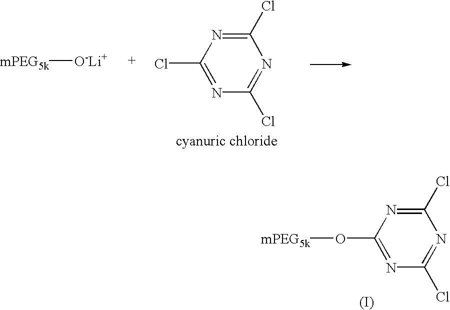

In a round-bottom flask, 10 grams of methoxy-PEG ₅ₖ(mPEG ₅ₖ)(2 mmole) was added to 50 ml of anhydrous toluene. Toluene (25 ml) was removed during a 2-hour distillation. The residue was cooled to 45° C. To the resulting solution was added dropwise n-butyllithium (2.5 M in hexane) containing 0.2 wt % of 1,10 phenanthroline. When the solution turned from yellow to brown-orange, the addition was terminated. The resulting solution was then added to a solution of cyanuric chloride (3.6 gram, 20 mmole) in anhydrous toluene. The final solution was stirred overnight and then added directly to 200 ml of ethyl ether. The precipitate (I) was collected by filtration, washed with fresh ether, and dried under vacuum. ¹H NMR(DMSO-d₆): δ3.50 (br m, PEG), 4.39 (t, CH₂O).

This example illustrates a method of conjugating a simple mPEG backbone functionalized with hydroxyl to cyanuric halide, wherein the hydroxyl group is first converted to an alkoxide.

### EXAMPLE 2

### Synthesis of 2-mPEGyloxy₅ₖ-4,6-di-(N-methylamino)-1,3,5-triazine (II)

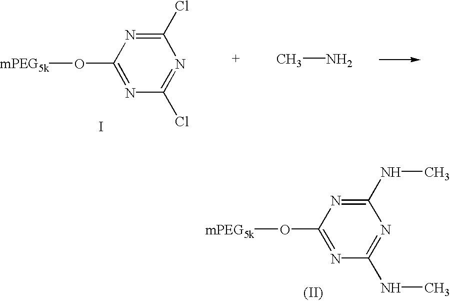

In a round-bottom flask, 4.5 grams of 2-mPEGylOXY₅ₖ-4,6-dichloro-1,3,5-triazine (I) was dissolved in 40 ml of anhydrous toluene. To the solution was added 12 ml of methyl amine (2M in THF). The solution was stirred overnight at 30° C. The solution was filtered and then added directly to 200 ml of ethyl ether. The precipitate 2-nmPEGyloxy₅ₖ-4,6-di-(N-methylamino)-1,3,5-triazine (II) was collected by filtration and dried under vacuum. ¹H NMR(DMSO-d₆): δ3.50 (br m, PEG), 7.0 (m, NH), 4.26 (t, CH₂O), 2.73 (s, CH₃).

This example illustrates reaction of a polymer-derivatized dihalo triazine precursor with an alkyl amine to replace the remaining halogen atoms with a substituted amino group.

### EXAMPLE 3

### Synthesis of 2-mPEGyloxy_{5K}-4,6-di-(N-methyl-N-hydroxymethylamino)-1,3,5-triazine

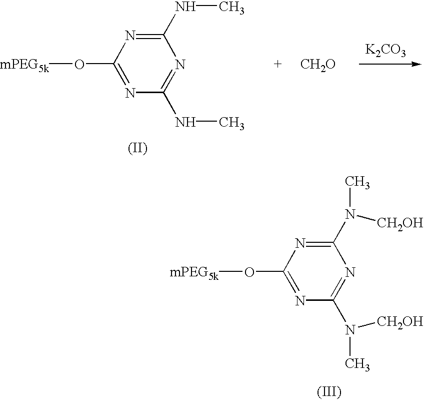

In a round-bottom flask, 2 grams of 2-mPEGyloxy₅ₖ-4,6-di-(N-methylamino)-1,3,5-triazine (II)was dissolved in 30 ml of an aqueous solution of potassium carbonate (50 mM). To the solution was added 15 ml of aqueous formaldehyde solution (37 wt %). The solution was stirred overnight at 45° C. The solution was then cooled to room temperature, and diluted with 5 wt % NaCl solution (90 ml). The resulting solution was extracted with methylene chloride. The organic phase was dried over sodium sulfate and filtered. The filtrate was concentrated under vacuum and the residual syrup was added to 50 ml of ethyl ether. The resulting precipitate was collected by filtration and dried under vacuum to give 2-mPEGyloxy₅ₖ-4,6-di-(N-methyl-N-hydroxymethylamino)-1,3,5-triazine (III). ¹H NMR(DMSO-d₆): δ3.50 (br m, PEG), 4.34 ( t, CH₂O), 3.03 (s, CH₃),4.99 (d, CH₂OH), 5.68 (t, CH₂OH).

In this example, the substituted amino groups added to the triazine ring in Example 2 are further modified by reaction with formaldehyde.

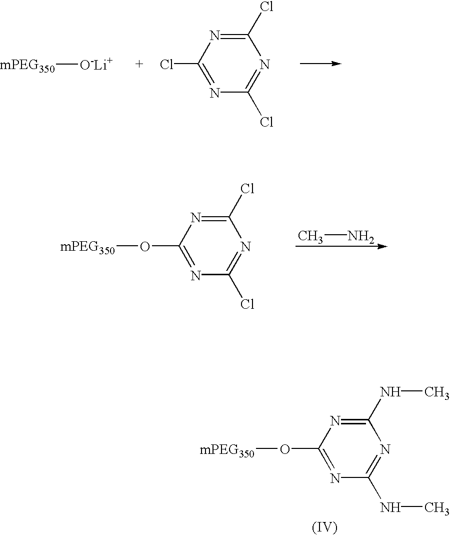

### EXAMPLE 4

### Synthesis of 2-mPEGyloxy₃₅₀-4,6-di-(N-methylamino)-1,3,5-triazine (IV)

In a round-bottom flask, 10 grams of methoxy-PEG ₃₅₀ was added to 100 ml of anhydrous toluene. Toluene (40 ml) was removed during a 2-hour distillation. The residue was cooled to 45° C. To the resulting solution was added dropwise butyl-lithium (2.5 M in hexane) containing 0.2 wt % of 1,10 phenanthroline. When the solution turned from yellow to brown-orange, the addition was terminated. The resulting solution was then added to the solution of cyanuric chloride (30 g) in anhydrous toluene (120 ml) and the resulting solution was stirred overnight. The solution was filtered and 400 ml of methyl amine (2M in THF) was added to it. The resulting mixture was stirred overnight at 30° C. The solution was then filtered and the filtrate was concentrated to give 2-mPEGyloxy₃₅₀-4,6-di-(N-methylamino)-1,3,5-triazine (IV) as an oil. ¹H NMR(DMSO-d₆ ): δ3.50 (br m, PEG), 7.0 (m, NH), 4.26 (t, CH₂O), 2.73 (s, CH₃).

In this example, a smaller molecular weight mPEG is utilized in the reactions outlined in Examples 1 and 2 above.

### EXAMPLE 5

### Synthesis of 2-mnPEGyloxY₃₅₀-4,6-di-(N-methvl-N-hydroxymethylamino)-1,3 5-triazine

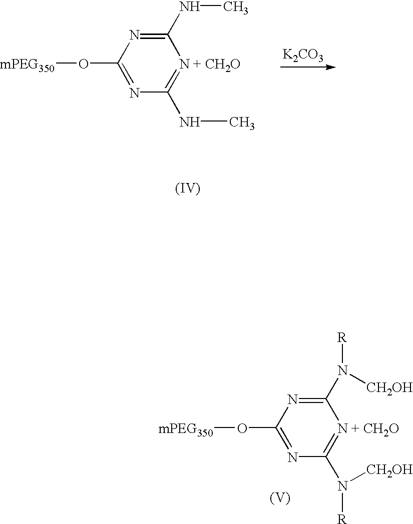

In a round-bottom flask, 0.5 gram of 2-mPEGyloxy₃₅₀-4,6-di-(N-methylamino)-1,3,5-triazine (IV) was dissolved in 15 ml of aqueous solution of potassium carbonate (50 mM). To the solution was added 15 ml of aqueous formaldehyde solution (37 wt %) and the solution was stirred overnight at 50° C. The solution was then cooled to room temperature, and diluted with 5 wt % NaCl solution (60 ml). The resulting solution was extracted with methylene chloride. The organic phase was dried over sodium sulfate and filtered. The product, 2-mPEGyloxy₃₅₀-4,6-di-(N-methyl-N-hydroxymethylamino)-1,3,5-triazine (V), was collected by removal of solvent under vacuum. ¹H NMR(DMSO-d₆): δ3.50 (br m, PEG), 4.34 ( t, CH₂O), 3.03 (s, CH₃), 4.99 (d, CH₂OH), 5.68 (t, CH₂OH ).

This example further modifies the substituted amino groups on the triazine ring of the polymer conjugate created in Example 4 by reaction with formaldehyde.

### EXAMPLE 6

### Synthesis of (VIII), a 4-arm PEG₂₀ₖanalogue of (III)

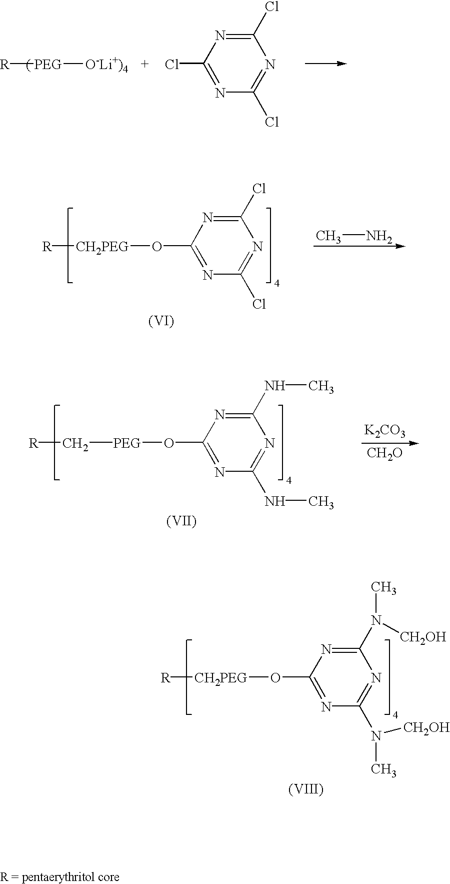

In a round-bottom flask, 10 grams of 4-arm-PEG ₂₀ₖ(polyethoxylated pentaerytliritol) was added to 160 ml of anhydrous toluene. Toluene (20 ml) was removed during a 2-hour azeotropic distillation. The residue was cooled to 40° C. To the resulting solution was added dropwise fresh butyl-lithium (2.5 M in hexane) containing 0.2 wt % of 1,10 phenanthroline. During addition of butyl-lithium, the solution became very viscous and some gel clusters appeared. When the mixture turned from yellow to brown-orange, the addition was terminated. To the resulting mixture was then added a solution (25 ml) of cyanuric chloride (3 grams) in anhydrous toluene. The final mixture was stirred overnight. The resulting solution was then added directly to 200 ml of ethyl ether. The precipitate (VI) was collected by filtration, washed with fresh ether, and dried under vacuum.

In a round-bottom flask, 5 grams (VI) was dissolved in 50 ml of anhydrous toluene. To the solution was added 12 ml of methyl amine (2M in THF). The solution was stirred overnight at 30° C. The solution was filtered through a fine filter and then added directly to 200 ml of ethyl ether. The precipitate (VII) was collected by filtration and dried under vacuum. ¹H NMR(DMSO-d₆): δ3.50 (br m, PEG), 7.0 (m, NH), 4.26 (t, CH₂O), 2.73 (s, CH₃).

In a round-bottom flask, 1 gram of (VII) was dissolved in 10 ml of aqueous solution of potassium carbonate (50 mM). To the solution was added 10 ml of aqueous formaldehyde solution (37 wt %). The solution was stirred overnight at 45° C. The solution was then cooled to room temperature, and diluted with 5 wt % NaCl solution (60 ml). The resulting solution was extracted with methylene chloride. The organic phase was dried over sodium sulfate and filtered. The filtrate was concentrated under vacuum and the residual syrup was added to 50 ml of ethyl ether. The resulting precipitate was collected by filtration and dried under vacuum to give (VIII). ¹H NMR(DMSO-d₆): δ3 .50 (br m, PEG), 4.34 (t, CH₂), 3.03 (s, CH₃), 4.99 (d, CH₂OH),5.68 (t, CH₂OH).

This example utilizes a multi-arm polymer backbone comprising a polyol core in the general reaction scheme outlined in Examples 1-3 above.

### EXAMPLE 7

### Synthesis of (XI), a homobifunctional PEG₃₄₀₀ analogue of (III)

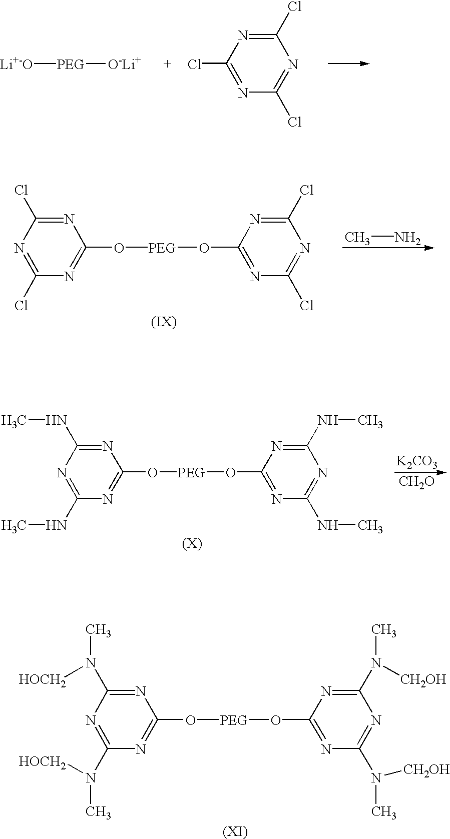

In a round-bottom flask, 20 grams of PEG ₃₄₀₀ was added to 200 ml of anhydrous toluene. Toluene (40 ml) was removed during a 2-hour azeotropic distillation. The residue was cooled to 40° C. To the resulting solution was added dropwise fresh butyl-lithium (2.5 M in hexane) containing 0.2 wt % of 1,10 phenanthroline. During addition of butyl-lithium, the solution became viscous and some gel clusters appeared. When the mixture turned from yellow to brown-orange, the addition was terminated. To the resulting mixture was then added a solution (100 ml) of cyanuric chloride (22 grams) in anhydrous toluene. The final mixture was stirred overnight. The resulting solution was filtered and then added to 700 ml of ethyl ether. The precipitate (IX) was collected by filtration, washed with fresh ether, and dried under vacuum.

In a round-bottom flask, 10 grams of (IX) was dissolved in 100 ml of anhydrous toluene. To the solution was added 20 ml of methyl amine (2M in THF). The solution was stirred overnight at 40° C. The solution was filtered through a fine filter and then added directly to 600 ml of ethyl ether. The precipitate (X) was collected by filtration and dried under vacuum. ¹H NMR(DMSO-d6): δ3.50 (br m, PEG), 7.0 (m, NH), 4.26 (t, CH₂O), 2.73 (s, CH₃).

In a round-bottom flask, 3.9 grams (X) was dissolved in 20 ml of aqueous potassium carbonate (50 mM). To the solution was added 20 ml of aqueous formaldehyde solution (37 wt %). The solution was stirred overnight at room temperature. The solution was diluted with 5 wt % NaCl solution (80 ml). The resulting solution was extracted with methylene chloride. The organic phase was dried over sodium sulfate and filtered. The filtrate was concentrated under vacuum and the residual syrup was added to 100 ml of ethyl ether. The resulting precipitate (XI) was collected by filtration and dried under vacuum. ¹H NMR(DMSO-d₆): 67 3.50 (br m, PEG), 4.34 ( t, CH₂O), 3.03 (s, CHhd 3), 4.99 (d, CH₂OH), 5.68 (t, CH₂OH).

Example 7 utilizes a bifunctional PEG backbone in the general reaction scheme outlined in Examples 1-3 above.

### EXAMPLE 8

### Synthesis of (2-N-mPEG₅ₖ-2-N-hydroxymethylamino)-4,6-di(N-methyl-N-hydroxymethyl)-1,3,5-triazine (XIV)

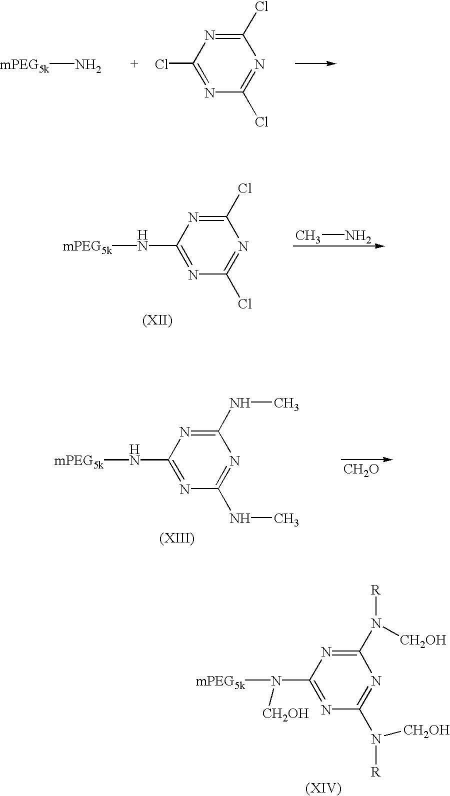

In a round-bottom flask, 1 gram of methoxy-PEG amine 5KDa (0.2 mmole) was added to 60 ml of anhydrous toluene. Toluene (40 ml) was removed during a 2-hour azeotropic distillation. The residue was cooled to 35° C., and then added to a solution (10 ml) of cyanuric chloride (0.37 gram) in anhydrous toluene. The final solution containing (XII) was stirred at room temperature for 4 hours. To the solution was added 10 ml of methyl amine (2M in THF). The solution was transferred into a heavy-duty glass tube with cap and stirred overnight over oil bath at 80° C. The solution was filtered, the solvent was condensed and then added directly to 100 ml of ethyl ether. The precipitate (XIII) was collected by filtration and dried under vacuum. ¹H NMR(DMSO-d₆): 67 3.50 (br m, PEG), 2.67 (s, CH₃).

In a round-bottom flask, 1 grams of (XIII) was dissolved in 10 ml of aqueous solution of potassium carbonate (50 mM). To the solution was added 10 ml of aqueous formaldehyde solution (37 wt %). The solution was stirred overnight at 50 ° C. The solution was then cooled to room temperature, and diluted with 5 wt % NaCl solution (45 ml). The resulting solution was extracted with methylene chloride. The organic phase was dried over sodium sulfate and filtered. The filtrate was concentrated under vacuum and the residual syrup was added to 50 ml of ethyl ether. The resulting precipitate (XIV) was collected by filtration and dried under vacuum. ¹H NMR(DMSO-d₆): 67 3.50 (br m, PEG), 3.01 (s, CH₃), 4.97 (d, CH₂OH).

This example illustrates the use of a polymer backbone functionalized with an amino group in the general reaction scheme outlined in Examples 1-3.

### EXAMPLE 9

### Stability study of (III), (V), and (XIV) in D₂O

Compound III, V, and XIV (6 mg) were separately dissolved in 0.75 ml of D₂O and stored at 37° C. The methylene peak disappearance as well as methyl peak shift were monitored by 300 MHz NMR over time. The half-life of the formaldehyde release reaction was monitored using first order kinetics. The half-life (t_{1/2) of both III and V was} 81 hours in D₂O, while that of XIV was 19.4 hours. The MALDI-TOF spectrum of hydrolyzed V showed that there was no detectable dimerization.

This example indicates that the polymer conjugates of trimelamol provided by the invention release formaldehyde in solution at physiological temperatures, which is believed to be a possible mechanism of action of the parent compound, trimelamol. Further, this example indicates that the polymer conjugates of trimelamol are more stable to dimerization than the parent compound.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A polymer conjugate of a triazine derivative comprising a water soluble and non-peptidic polymer backbone covalently attached to a non-heteroatom position of a triazine derivative comprising an s-triazine ring or an as-triazine ring.

2. The polymer conjugate of claim 1, wherein the triazine derivative comprises a 1,3,5-triazine ring, a 1,2,4-triazine ring or a benzotriazine ring.

3. The polymer conjugate of claim 1, wherein the triazine derivative is substituted at one or more non-heteroatom positions with a substituent selected from the group consisting of amino, substituted amino, aryl, and substituted aryl.

4. The polymer conjugate of claim 1, wherein the polymer backbone is selected from the group consisting of poly(alkylene glycol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), and copolymers, terpolymers, and mixtures thereof.

5. The polymer conjugate of claim 1, wherein the polymer backbone is poly(ethylene glycol).

6. The polymer conjugate of claim 5, wherein the poly(ethylene glycol) has an average molecular weight from about 100 Da to about 100,000 Da.

7. The polymer conjugate of claim 1, wherein the polymer backbone has about 2 to about 300 termini.

8. The polymer conjugate of claim 1, wherein the polymer backbone is covalently bonded to the structure: 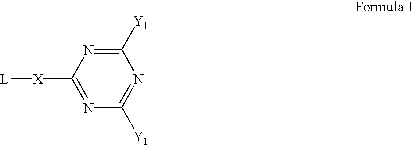
wherein:
L is the point of attachment to the polymer backbone;
X is a linker; and
Y₁ and Y₂ are each independently amino, substituted amino, C1-6alkyl, substituted C1-6alkyl, aryl, or substituted aryl.

9. The polymer conjugate of claim 8, wherein X is O or NR₂, wherein R₂ is H, C1-6alkyl, or substituted C1-6alkyl.

10. The polymer conjugate of claim 8, wherein Y₁ and Y₂ are each NRR₁, wherein R is C1-6alkyl, substituted C1-6alkyl, or an electron withdrawing group, and R₁ is H, C1-6alkyl, or substituted C1-6alkyl.

11. The polymer conjugate of claim 10, wherein R is methyl and R₁ is —CH₂OH. CH₂OH.

12. The polymer conjugate of claim 8, having the structure: 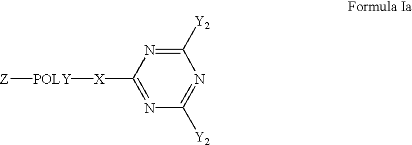
wherein POLY is a water soluble and non-peptidic polymer backbone, Z is a capping group, and X, Y₁ and Y₂ are as defined above.

13. The polymer conjugate of claim 12, wherein POLY is selected from the group consisting of poly(alkylene glycol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), and copolymers, terpolymers, and mixtures thereof.

14. The polymer conjugate of claim 12, wherein POLY is poly(ethylene glycol).

15. The polymer conjugate of claim 12, wherein X is O or NR₂, wherein R₂ is H, C1-6alkyl, or substituted C1-6alkyl.

16. The polymer conjugate of claim 12, wherein Z is selected from the group consisting of alkoxy, hydroxyl, protected hydroxyl, active ester, active carbonate, acetal, aldehyde, aldehyde hydrates, alkenyl, acrylate, methacrylate, acrylamide, active sulfone, amine, protected amine, hydrazide, protected hydrazide, thiol, protected thiol, carboxylic acid, protected carboxylic acid, isocyanate, isothiocyanate, maleimide, vinylsulfone, dithiopyridine, vinylpyridine, iodoacetamide, epoxide, glyoxals, diones, mesylates, tosylates, and tresylate.

17. The polymer conjugate of claim 12, wherein Z has the structure: 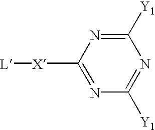
wherein X′ is a linker, L′ is the point of attachment to POLY, and Y₁ and Y₂ are as defined above.

18. The polymer conjugate of claim 17, wherein X′ is O or NR₂, wherein R₂ is H, C1-6alkyl, or substituted C1-6alkyl.

19. The polymer conjugate of claim 17, wherein POLY is poly(ethylene glycol) and X and X′ are both O.

20. The polymer conjugate of claim 12, wherein POLY is poly(ethylene glycol) and Z is methoxy.

21. The polymer conjugate of claim 8, having the structure: 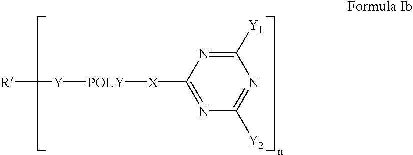
wherein:
n is an integer from 3 to about 100;
R′ is a central core molecule;
X and Y are each independently selected linkers;
each POLY is an independently selected water-soluble and non-peptidic polymer backbone; and
Y₁ and Y₂ are as defined above.

22. The polymer conjugate of claim 21, wherein n is about 3 to about 20.

23. The polymer conjugate of claim 21, wherein each X and Y are independently selected from the group consisting of O or NR₂, wherein R₂ is H, C1-6alkyl, or substituted C1-6alkyl.

24. The polymer conjugate of claim 21, wherein R′ is derived from a molecule selected from the group consisting of polyols, polyamines, and molecules having a combination of alcohol and amine groups.

25. The polymer conjugate of claim 21, wherein R′ is derived from a molecule selected from the group consisting of glycerol, glycerol oligomers, pentaerythritol, sorbitol, and lysine.

26. The polymer conjugate of claim 21, wherein each POLY is selected from the group consisting of poly(alkylene glycol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly N-acryloylmorpholine), and copolymers, terpolymers, and mixtures thereof.

27. The polymer conjugate of claim 21, wherein each POLY is poly(ethylene glycol).

28. The polymer conjugate of claim 21, having the structure: 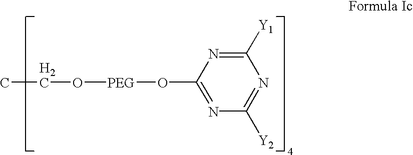
wherein PEG is poly(ethylene glycol) having an average molecular weight from about 100 Da to about 100,000 Da, and Y₁ and Y₂ are as defined above.

29. The polymer conjugate of claim 8, wherein the polymer backbone is bonded to the structure: 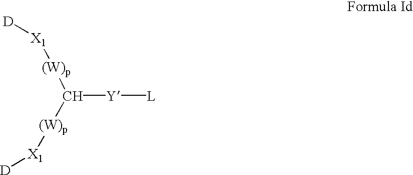
wherein X₁ and Y′ are independently selected linkers, L is the point of bonding to the polymer backbone, each p is independently 0 or 1, and each W is independently selected from the group consisting of —(CH₂)ₘ—, —(CH₂)ₘ—O—, —O—(CH₂)ₘ—, —(CH₂)ₘ—O₂C—CH₂CH₂—, and —(CH₂)ₘ—O—(CH₂)ᵣ—, wherein m and r are independently 1—10, and each D is a triazine derivative having the structure: 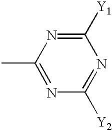
wherein Y₁ and Y₂ are as defined above.

30. The polymer conjugate of claim 29, wherein X₁ and Y′ are independently selected from the group consisting of O or NR₂, wherein R₂ is H, C1-6alkyl, or substituted C1-6alkyl.

31. The polymer conjugate of claim 1, wherein the polymer backbone has the structure: 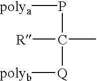
wherein
Polyₐ and poly_{b} are water-soluble and non-peptidic polymer backbones;
R″ is a nonreactive moiety; and
P and Q are nonreactive linkages.

32. The polymer conjugate of claim 31, wherein polyₐ and Poly_{b} are both methoxy poly(ethylene glycol).

33. The polymer conjugate of claim 31, wherein R″ is H, methyl or a water-soluble and non-peptidic polymer backbone.

34. The polymer conjugate of claim 1, wherein the polymer backbone comprises methoxy poly(ethylene glycol) disubstituted lysine.

35. A method of forming a polymer conjugate of a triazine derivative, comprising:
providing a water soluble and non-peptidic polymer backbone bonded to a functional group reactive with cyanuric halide;
reacting the polymer backbone with cyanuric halide, the cyanuric halide comprising a trihalo-substituted triazine ring, to form a dihalotriazine intermediate having a polymer backbone covalently attached at one of the non-heteroatom positions of the triazine ring; and
replacing each of the two remaining halogen atoms of the dihalotriazine intermediate with a functional group.

36. The method of claim 35, wherein the cyanuric halide has the structure: 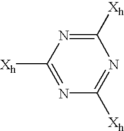
wherein each Xₕ is halogen.

37. The method of claim 36, wherein Xₕ is chlorine.

38. The method of claim 35, wherein the functional group of the water-soluble and non-peptidic polymer backbone is selected from the group consisting of hydroxyl, alkoxide, and amine.

39. The method of claim 35, wherein said step of reacting the polymer backbone with the cyanuric halide occurs in the presence of a solvent selected from the group consisting of toluene, tetrahydrofuran, and dioxane.

40. The method of claim 35, wherein the polymer backbone is selected from the group consisting of poly(alkylene glycol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), and copolymers, terpolymers, and mixtures thereof.

41. The method of claim 35, wherein the polymer backbone is poly(ethylene glycol).

42. The method of claim 41, wherein the poly(ethylene glycol) has an average molecular weight from about 100 Da to about 100,000 Da.

43. The method of claim 35, wherein said step of replacing each of the two remaining halogen atoms of the dihalotriazine intermediate with a functional group comprises replacing each halogen atom with a functional group selected from the group consisting of amino, substituted amino, C1-6alkyl, substituted C1-6alkyl, aryl, or substituted aryl.

44. The method of claim 35, wherein said replacing step comprises reacting the triazine intermediate with an alkyl amine to form a diamino-substituted triazine polymer conjugate having the structure: 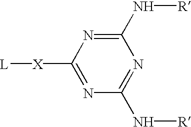
wherein L is the point of attachment to the polymer backbone, X is a linker, and R′ is alkyl.

45. The method of claim 44, wherein the alkyl amine is methyl amine and R′ is methyl.

46. The method of claim 44, wherein the reaction with the alkyl amine occurs in the presence of a solvent selected from the group consisting of toluene, tetrahydrofaran, dioxane, acetonitrile, methylene chloride, and chloroform.

47. The method of claim 44, further comprising reacting the diamino-substituted triazine polymer conjugate with aqueous formaldehyde in the presence of an alkali metal carbonate to form a disubstituted amino triazine polymer conjugate having the structure: 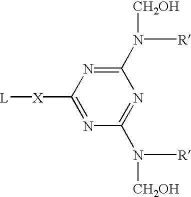
wherein L, X and R′ are as defined above.

48. A method of treating cancer in a mammal, the method comprising administering to the mammal a therapeutically effective amount of a polymer conjugate of a triazine derivative comprising a water soluble and non-peptidic polymer backbone covalently attached to a non-heteroatom position of a triazine derivative comprising an s-triazine ring or an as-triazine ring.

49. The method of claim 48, wherein the triazine derivative comprises a 1,3,5-triazine ring, a 1,2,4-triazine ring or a benzotriazine ring.

50. The method of claim 48, wherein the triazine derivative is substituted at one or more non-heteroatom positions with a substituent selected from the group consisting of amino, substituted amino, aryl, and substituted aryl.

51. The method of claim 48, wherein the polymer backbone is selected from the group consisting of poly(alkylene glycol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), and copolymers, terpolymers, and mixtures thereof.

52. The method of claim 48, wherein the polymer backbone is poly(ethylene glycol).

53. The method of claim 52, wherein the poly(ethylene glycol) has an average molecular weight from about 100 Da to about 100,000 Da.

54. The method of claim 48, wherein the polymer backbone is covalently bonded to the structure: 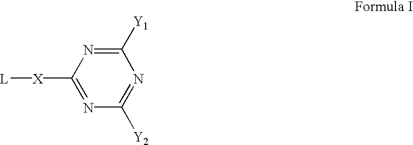
wherein:
L is the point of attachment to the polymer backbone;
X is a linker; and
Y₁ and Y₂ are each independently amino, substituted amino, C1-6alkyl, substituted C1-6alkyl, aryl, or substituted aryl.

55. The method of claim 54, wherein X is O or NR₂, wherein R₂ is H, C1-6alkyl, or substituted C1-6alkyl.

56. The method of claim 54, wherein Y₁ and Y₂ are each NRR₁, wherein R is C1-6alkyl, substituted C1-6alkyl, or an electron withdrawing group, and R₁ is H, C1-6alkly, or substituted C1-6alkyl.

57. The method of claim 54, wherein R is methyl and R₁ is —CH₂OH.

58. The method of claim 48, wherein said administering step comprises administering the compound buccally, subcutaneously, transdermally, intramuscularly, intravenously, orally, or by inhalation.
